(19) United States
(12) Patent Application Publication
(10) Pub. No.: US 2015/0111790 A1
(43) Pub. Date: Apr. 23, 2015

(54) TESTING DEVICE

(71) Applicants: Christopher Ategeka, Oakland, CA (US); Anwaar Al-Zireeni, Antioch, CA (US)

(72) Inventors: Christopher Ategeka, Oakland, CA (US); Anwaar Al-Zireeni, Antioch, CA (US)

(21) Appl. No.: 13/261,983

(22) PCT Filed: Jan. 30, 2014
(86) PCT No.: PCT/US2014/000015

§ 371 Date: Nov. 3, 2014

Related U.S. Application Data
(60) Provisional application No. 61/759,395, filed on Jan. 31, 2013.

(51) Int. Cl. G01N 33/569; C12Q 1/70; G01N 21/78

(52) U.S. Cl. CPC G01N 33/56988; G01N 21/78; C12Q 1/703; G01N 2333/162
USPC 506/39; 435/287.1; 435/287.2

(57) ABSTRACT

The invention is a device for testing an individual for the presence of a entity. The entity can be a pathogen, toxin or drug. The device is adapted to collect from the individual the biological sample to be tested. The test device comprises noble metal nanoparticles (i.e. gold, silver or copper) to detect the entity. Test results are read from a window in the device. The current visual limit using aggregated noble metal nanoparticles is a clump having a diameter in a range of about 50 nm to about 75 nm. One or more ligands specific for the entity are attached to two or more metal nanoparticles. Each individual nanoparticle has a diameter in a range of about 15 nm to about 35 nm. Ligands attached to the nanoparticles and specific for the entity can be for example antibodies, antibody fragments, oligonucleotides, and aptamers. Different ligands specific for different sites on the entity may be used in order to increase the aggregation potential of the nanoparticles in the presence of the target. The testing is conducted within the device after sample collection. The results of assay are available promptly. Conditions that can be tested include for example viral infections, bacterial infections, exposure to toxins, and drug use. Detection of Human Immunodeficiency Virus (HIV) is described by example.

### TECHNICAL FIELD

The technical field is a device that collects, tests, and reports detection of a target entity in a biological sample.

### BACKGROUND OF THE INVENTION

Detection of pathogenic agents, toxins, and drugs in biological samples is continually being improved and refined. Home test kits are sold to test for a wide range of conditions including pregnancy and sexually transmitted diseases (STDs). In many cases, the knowledge learned from a home test is valuable for the purpose of treatment and to reduce the risk of transmission of an infectious agent to others.

There are many viral, bacterial, and pathogenic agents for which there are no easy testing methods. Individuals who suspect contact with an infective agent, or who are symptomatic of infection are required to seek diagnosis from medical professionals at medical facilities. With some conditions, notably HIV and other viral infections, standard testing modalities preclude early detection, and in the case of HIV, to test for the presence of antibodies to HIV in body fluid and individual must wait until about 6 months after a suspected infection date. Individuals are therefore often discouraged from seeking diagnosis due to the requirement of waiting, and possibly due also to expense where the test must be performed at a health care facility. With most of these conditions prompt treatment after contact or infection will increase the likelihood of successful treatment. With infectious conditions, prompt diagnosis and treatment can reduce the spread of the condition to others.

In particular, detection of human immune-deficiency virus (HIV) infection is critical to both prevention and treatment strategies. However, even 30 years after the beginning of the pandemic, the laboratory tools available to test for HIV infection are imperfect, and very few individuals receive diagnosis and care after recent infection. Over 33.4 million people worldwide suffer from Human Immunodeficiency Virus (HIV), while 2 million die from its progression to Acquired Immune Deficiency Syndrome (AIDS), annually. Six million people in developing countries are in urgent need of antiretroviral therapy, but only 700,000 are currently receiving effective treatments due to limited accessibility and high costs.

Methods of testing for detectable biological entities in biological samples are being explored by a number of groups. Many research groups focus largely on optimizing and increasing the capabilities of existing technology for laboratory and large-scale diagnostic use, for use in medical facilities by medical professionals.

With regard to viral infections including HIV, some groups have developed kits using antibody detection for home screening tests, but these kits require the waiting period of about 6 months for the antibodies to appear in the infected individual. The drawback of using as a target for a detection assay an antibody specific for the infective agent is that the human body presents antibodies to many infective agents only about 4 to 6 months after infection. Thus, detection of antibodies specific for the infective agent is not possible for at least 4 to 6 months after infection of the individual. The situation is particularly acute with infants for whom it would be extremely desirable to detect an infectious disease early. Infants do not make antibodies to pathogenic infective agents until about one year after infection.

Early detection of pathogenic agents would serve to facilitate both early treatment and reduced transmission of those agents to others. It would be a great advantage to the medical community to provide early detections methods for all pathogenic agents including viral, and bacterial agents, and toxins and drugs.

### SUMMARY OF THE INVENTION

The invention is a testing device, kit, assay and method that provide an individual the opportunity to test for the presence of a target entity in a biological sample. The testing device is adapted to contain a biological sample collected from an individual. The testing device includes the reagents to assay the collected biological sample to detect a target entity of interest. The assay is a nanoparticle-based colorimetric assay that is visually reported from a results window on or in the test device. The result can be, for example, colorimetric or quantitative. The assay is conducted and the results are reported soon after the biological sample is collected. For infectious diseases, the test device can have a neutralizing function so that the device does not carry live infectious entities that can infect others.

The assay employs a colorimetric nanoparticle approach to detecting a target entity in a biological sample. The method of conducting the assay includes the steps of collecting a biological sample from an individual. The sample is collected and contained in a chamber within the testing device. Also within the testing device are the reagents necessary for conducting the colorimetric nanoparticle-based detection assay. Reagents that may be used include a plurality of noble metal nanoparticles having ligands conjugated on their surfaces. The ligands are specific for binding a target entity, or a component of a target entity, in the biological sample being tested. The reagents also include solutions and buffers for conducting the assay, one or more crowding agents if necessary, reagents to form a control assay, and any other reagent necessary for conducting the colorimetric nanoparticle-based detection assay. After the biological sample is collected, the collection chamber may be closed. The sample can be prepared prior to mixing with agents, for example by separating the plasma from other constituents of whole blood. The prepared sample can be mixed directly with reagents within an assay chamber in the device. When the assay is complete, the results of the assay are revealed in a results window on the device. The colorimetric assay is read with the naked eye. Options for display of the results include presenting a color or color change in the results window. Quantitation may be measured in terms of intensity of color, or hue, and can be digitized to a numerical readout if desired. The color of the colorimetric assay can be “read” as a numerical quantity or a color, or both, for example using a sensor. Quantitation can include measuring the absorbed or reflected light both in terms of a wavelength of light and intensity, or it can include measuring an amount of nanoparticles bound to the target entity.

The device is designed to provide the individual the opportunity to perform all the steps of the method without the assistance of a health care professional. Accordingly, the individual can collect the sample themselves, place the sample in a chamber having an opening in the test device, seal the chamber shut, manipulate components on and in the device to allow contact of the sample with a solution comprising the reagents for a detection assay, and read the results displayed as a color in a window of the device, or a digitized value or quantitation. The results of the assay, if digitized may also be reported to a smart phone or other smart device. Instructions for use of the device and the kit components can be included in the box with the device and other components. The instructions will describe such details as how to interpret the color displayed in the results window of the device, or a numerical value representing detection of a target entity. Generally, the sample will be considered positive for the target entity if a certain color is shown in the window (for example with a showing of blue if gold nanoparticles are used). Numerical values and their meanings would be defined by calibration and described in the instructions for use.

The testing device, kit, assay and method of this invention can be used for detecting all types of pathogens, including viral and bacterial agents and toxins and drugs. All target entities of pathogenic agents that are detectable in biological samples can form the basis of a design of a testing device, kit, and assay as described herein. Elements of the device and invention that vary from target entity to target entity have to do with the nature of the target entity, the ligands used to detect that entity, and biological fluid or tissue from which that target entity is assayed, the optimal time after suspected infection or contact that an individual can collect a biological sample, and other idiosyncratic details inherent in the target entity being detected.

The colorimetric detection assay is based on the principles that noble metal nanoparticles that aggregate at least as close as the diameter of the nanoparticles resonate at a different plasmonic resonance frequency than unbound nanoparticles, non-aggregated nanoparticles of the same size and material. Thus using nanoparticles tagged with ligand specific for binding the target entity (or a component of a target entity) can facilitate detection of the target entity. A color shift occurs in the solution when the nanoparticle conjugates come together in close proximity and form a clump. The color shift indicates the presence of the target entity in the sample. Nanoparticles in close proximity with each other reflect a different frequency of visible light than the non-aggregated metal nanoparticles reflect. If there is no color shift, the metal nanoparticle ligand conjugate remains unbound to any target and therefore the biological sample is negative for the target entity.

The biological sample collected from the individual can be fluidic or non-fluidic. Typically, collecting the sample involves placing a small amount of the individual's body fluid in a reservoir having an opening in the test device. A body fluid collected from the individual can be, for example, blood, lymph, saliva, or urine. The sample can also be a non-fluidic material, for example a viscous or solid material. Non-fluidic body materials collectable by the individual can include, for example, skin, tissue from an open wound, a scab, puss, hair, secretions, mucous, or excrement. The amount collected will be the amount needed for conducting the assay, and also the amount that is reasonably collectable. The target entity will be found in a biological sample collected from an individual and the biological sample can be selected from blood, semen, vaginal secretion, breast milk, amniotic fluid, or lymph. The target entity found in the biological sample can be, for example, a cell, protein, peptide, hormone, nucleic acid, a virus, a bacterium, an organic molecule, a protein, a peptide, a nucleic acid, a lipid, a fatty acid, a carbohydrate, a drug, a hormone, a cell, an element, a toxin, a chemical, a metabolite, or a complex comprising two or more of any of the aforementioned items.

Thus, for example, in the case of HIV infection, very low levels of HIV may be present in the blood 7 to 10 days after infection, on the order of 1 HIV viron per 50 ul of blood. Accordingly, about 50 ul of blood will be collected. The volume of a drop of blood depends on the size of the drop, and is reported to be anywhere from 20 ul to 50 ul. If two drops of blood are collected in the device, the volume collected would be in a range from about 40 to about 100 ul. Different testing situations and different conditions may require more or less fluid or mass of biological sample.

A kit for testing a biological sample collected from an individual can comprise a testing device having components for sample collection, testing, and displaying test results. The testing components can include a solution having reagents for conducting the detection assay and comprising a plurality of noble metal nanoparticles conjugated to ligands specific for the target entity. The kit also usually includes a container for the kit contents and instructions for use. The test device can include a chamber for collecting the biological sample from the individual, and a chamber having pre-loaded reagents for conducting the assay. The test device also includes a window from which the individual can read the test results. The testing device can include quantitation. In the case of an infectious pathogen, the testing device can further include a system, reagent or mechanical feature to neutralize or inactivate the live pathogen, and also to isolate it from contact after completion of sample collection.

### BRIEF DESCRIPTION OF THE DRAWINGS

FIG. 1 is a cartoon depiction of an HIV viron.

FIG. 2 is a chart of time vs. detectable HIV subpart.

FIG. 3 is a schematic drawing of a testing device.

### DETAILED DESCRIPTION OF THE INVENTION

The testing device, assay and kit are for detecting an entity in a sample taken from an individual. The testing assay can be self-administered by the individual who provides the biological sample to be tested, and does not require a lab to collect or prepare the sample, or a clinician to read and interpret the test results. With regard to HIV testing, the sensitive nature of the detection assay provides the opportunity to test an individual for the virus within 5 days after suspected contact.

The testing device, test kit, assay and method of the invention comprise use of a solution-based noble metal nanoparticle colorimetric detection. The nanoparticles are conjugated to ligands specific for target entities. The ligands can be specific for the target entity, or a component of the target entity, and there can be nanoparticles conjugated to the same or different ligands having different specificities. In the design of the ligands for a particular assay, the goal is to facilitate aggregating of the nanoparticles in the presence of target entity that occurs when multiple nanoparticles bind (through their conjugated ligands) a target entity or its components. The aggregation of the metal nanoparticles causes a color shift in the solution, for example with gold nanoparticles the solution is red in a solution having non-aggregated particles and blue when the nanoparticles aggregate.

For example, a solution of gold nanoparticles having a uniform size in a range from about 10 nm to about 30 nm diameter, spaced in solution at a distance greater than any one diameter of any one nanoparticle, will have a red color to the naked eye. If those nanoparticles in solution are forced to aggregate and form clumps with other nanoparticles where each nanoparticle is closer than a diameter of anyone nanoparticle to one or more other nanoparticles, the solution changes color to blue. Additionally, in between the change in color from red to blue, shades of purple representing an amount of clumping can be observed in some cases.

The assay for this invention is conducted using noble metal nanoparticles and the principles of plasmonic resonance that apply to noble metal nanoparticles in solution and as aggregates. For one of the first groups to describe this technology, see “Selective Colorimetric Detection of Polynucleotides Based on the Distance-Dependent Optical Properties of Gold Nanoparticles” Robert Elghanian, James J. Storhoff, Robert C. Mucic, Robert L. Letsinger, Chad A. Mirkin, Science 277, 1078 (1997); DOI: 10.1126/science.277.5329.1078. Mirkin describes a highly selective, colorimetric polynucleotide detection method based on mercaptoal-kyloligonucleotide-modified gold nanoparticle probes. The unoptimized system can detect about 10 femtomoles of an oligonucleotide. For additional technical details on materials, reagents and conditions and other technical details for conducting the assays, see Chem. Rev. 2005, 105, 1547-1562 1547 “Nanostructures in Biodiagnostics”. Nathaniel L. Rosi and Chad A. Mirkin. Mirkin concludes that continued improvement of ligand design that allows for highly specific metal coordination will result in even more selective metal ion detection assays that implement nanostructured probes. See “Nanostructures in Biodiagnostics”. Chemical Reviews, 2005, Vol. 105, No. 4 1555. The Mirkin references are incorporated by reference in their entireties herein.

Solution-based nanoparticle assays use a colorimetric nanoparticle approach and take advantage of analyte-induced aggregation events that result in measurable changes and shifts of nanoparticle surface plasmon absorption bands. The simplicity of the colorimetric detection format pointed toward its use as a general method to detect wide varieties of analytes. See Haes, A. J.; Van Duyne, R. P. J. Am. Chem. Soc. 2002, 124, 10596, and Riboh, J. C.; Haes, A. J.; McFarland, A. D.; Yonzon, C. R.; Van Duyne, R. P. J. Phys. Chem. B 2003, 107, 1772. Lui and co-workers provided an example of colorimetric detection for toxins by implementing an oligonucleotide-assembled nanoparticle network to detect Pb(II) ions in aqueous media and lead-containing paint samples at concentrations as low as 100 nM. See Liu, J.; Lu, Y. J. Am. Chem. Soc. 2003, 125, 6642. Lui's group also used detected Hg toxin by inducing the aggregation of nanoparticles functionalized with appropriately designed chelating groups such as mercaptocarboxylic acids. Metal ions bridged carboxylate moieties of different gold nanoparticles, resulting in a concomitant colloidal color change from red to blue. See Liu, J.; Lu, Y. J. Am. Chem. Soc. 2003, 125, 6642. The Van Duyne and Lui references are incorporated by reference in their entirety.

Noble metal nanoparticles have been used to detect entities in biological samples based on the principles of plasmonic resonance that dictate a change in the absorbance frequency of visible light depending on the size of the noble metal particle. For example gold nanoparticles less than about 30 nm in diameter appear red in solution. Once the particle size increases beyond 60 nm, the aggregates cause the color of the solution to shift from red to blue. Between the red and blue shift, and related to the growing size of the aggregating nanoparticles, gradations of purple can be seen. The invention uses analyte formed by attaching ligands specific for the target entity to noble metal nanoparticles (ie. gold, silver or copper). For example, gold nanoparticles in solution each having a diameter of about 20-30 nm display a red color in solution. Nanoparticles tagged with ligands specific for a target will aggregate around the target if the target is present in the biological sample being tested, forming aggregates of ligand-conjugated nanoparticles all specific for the target. The aggregates of two, three, or more nanoparticles in a size range of about 20 nm to about 30 nm will form a clump greater than 60 nm. The plasmonic resonance of the gold nanoparticles then shifts the reflected visible light wavelength to blue and the solution appears blue to the naked eye.

Specific considerations in the design of the testing device and assay include details such as the fact that the signal for hybridization is governed by the optical properties of the nanoparticles, which depend in part on their spacing within the polymeric aggregate. Nanoparticle aggregates with inter-particle distances substantially greater than the average particle diameter appear red, but as the inter-particle distances in these aggregates decrease to less than approximately the average particle diameter, the color becomes blue. This shift, attributed to the surface plasmon resonance of the gold (Au), has been observed in both oligonucleotide and non-oligonucleotide-based strategies for organizing nanoparticles into aggregate structures and has been studied theoretically. Gold particles 13 nm in diameter have been used because they can be readily prepared with little deviation in size (2 nm) and exhibit a sharp plasmon absorption band (maximum absorbance at wavelength 520 nm).

According, elements and considerations of the assay of the invention include the size and quantity of the nanoparticles, the shape of the nanoparticles, the nature and character of the ligand conjugated to the nanoparticles, the target binding partner for the ligand, steric considerations in binding multiple nanoparticles to the same or different sites on the target, and feasibility of conducting the assay using a volume or mass of biological material collected within the assay chamber of the test device.

The assay includes a sample comprising a fluid or a non-fluid material from an individual. The sample can be a fluid and the fluid can be selected from blood, lymph, semen, vaginal fluid, breast milk, saliva, urine, cerebrospinal fluid, pleural fluid, pericardial fluid, amniotic fluid, synovial fluid, and interstitial fluid.

Pathogenic agents that can be detected in the biological sample are, for example, viral or bacterial agents. Other viral infective agents include, for example influenza, hepatitis, herpes, papilloma, adeno-associated virus, flavivirus, dengue virus, Japanese encephalitis virus, T-cell lymphotrophic virus, cytomegalovirus (CMV), Epstein-Barr virus, reovirus, vaccinia virus, parvovirus, feline leukemia virus, cauliflower mosaic virus, tomato bushy stunt virus and others, and other viral-caused or viral-related infections. Bacterial infections of all types can be detected, such as for example e-coli infections, sepsis, tetanus, and other common or not-so-common bacterium, including secondary infections that may develop after viral infections. In addition, conditions having detectable markers that can be the basis of a target entity in the testing device and assay of the invention can be detected. Such conditions can include, for example degenerative diseases such as Alzheimer's disease and other neurodegenerative diseases, and diseases involving muscular degeneration, proliferative conditions such as cancer, and restenosis, inflammatory conditions such as autoimmune diseases, hypersensitivity disorders, and allergies, metabolic conditions such as diabetes, and digestive disorders, and genetic conditions that manifest detectable markers. In addition to biological pathogens, the invention can detect toxins such as toxins from an environmental exposure, and drugs such as performance enhancing drugs. In general, any condition having a target entity that serves as a marker for infection or the condition, and for which a ligand can be developed and used as the basis to detect the target entity can be the basis of a specific testing device, kit, assay and method.

The target entity is present in the biological sample collected from the individual if the individual is positive for the condition being tested. Such target entities can be, for example markers of the condition, or they can be the actual toxin, drug, or pathogen being sought. Accordingly, the target entities (or components of the target entity) can be, for example, a nucleic acid, a ribo-nucleic acid, a polypeptide, a carbohydrate, a protein, a peptide, a polypeptide, an amino acid, a hormone, a steroid, a vitamin, an ion, a metabolite, a derivative, an analogue, a polysaccharide, a lipid, a lipopolysaccharide, a glycoprotein, a lipoprotein, a nucleoprotein, an oligonucleotide, an antibody, an immunoglobulin, a coagulation factor, a peptide hormone, a protein hormone, a non-peptide hormone, an interleukin, an interferon, a cytokine, a cell, a cell-surface molecule, a microorganism, a small organic molecule, a viron, a bacterium, a toxin, a drug, a cell, a cell membrane, a membrane fraction, a protein complex, an antigen, a hapten, a receptor, a macromolecule, or a molecular complex.

Other target entities are also possible to use in the assay. Thus, the entity targeted by the assay can be any entity found in a biological sample and considered a marker for the condition being sought. A target entity can be one or more molecules, peptides, oligonucleotides, small molecules, elements and other entities found within a biological sample and for which there is a ligand that can be conjugated to a noble metal nanoparticle.

The ligands are conjugated to the noble metal nanoparticles using known conjugation techniques. The ligands bind a target entity or a component of the target entity. The specific binding site or component of the target entity can vary from condition to condition and must be selected carefully. Considerations in selecting the target entity or the component of the target entity can include timing: the question can be asked of when, for example, in the lifecycle of the pathogen or the infection is that target entity present in the individual. Considerations can also include location: the question can be asked of where, for example, in the individual is that target entity found, and is it present in the type of biological sample that can be collected into the testing device. A ligand specific for a target entity or a component of a target entity can be, for example, an antibody, an antigen, a receptor, an aptamer, a protein, a polypeptide, small molecule a nucleic acid or any binding entity capable of binding the target entity or a component of the target entity. The ligand can form a binding pair with a binding member on the target, for example the binding pair can be an antigen and antibody-specific pair, biotin and avidin pair, carbohydrate and lectin pair, complementary nucleotide sequences, complementary peptide sequences, effector and receptor molecules, enzyme cofactor and enzymes, or enzyme inhibitors and enzymes. Many different ligands and binding pairs can be used to detect the targets in a single assay. Specific ligands or binding members conjugated to noble metal nanoparticles and designed to bind target entities can include ligands specific for binding and detecting any of the target entities or components of target entities listed, including also genes, coding sequences, codons, non-coding sequences, mitochondrial DNA, viral RNA, viral DNA, bacterial DNA, fungal DNA, mammalian DNA, cDNA, mRNA, RNA fragments, DNA fragments, oligonucleotides, synthetic oligonucleotides, modified oligonucleotides, single-stranded nucleic acids, double-stranded nucleic acids, complementary nucleotide sequences, branched DNA, components for copying a nucleic acid, amplicons, natural nucleic acids, synthetic nucleic acids, transcription factors, ligases, enzymes, and subunits of any of these.

Aspects of the invention that may vary from test device to test device and assay to assay are characterized essentially by the nature of the target entity to be detected and can include the following: the target entity, the ligands to bind the target entity, and the binding site on the target entity that the ligand can bind.

Other considerations can include whether the target entity binds with high affinity to a ligand, and whether there exist any steric hindrance or binding competition issues in that binding process. Also in designing ligands to be conjugated to nanoparticles, issues of whether the ligand can bind the target entity in a way that facilitates the nanoparticles to come in close proximity to one another is a concern so that a color shift occurs in the presence of the target entity. Finally, where more than one type of ligand is selected to bind a target entity or different components of the target entity, the question should be asked whether these ligands synergize to result in the desired color shift in the assay.

The sizes, shapes, and compositions of metal nanoparticles can be systematically varied to produce materials with specific emissive, absorptive, and light-scattering properties which make these materials ideal for multiplexed analyte detection; the composition of nanowires and nanotubes also can be controlled, thus allowing for measurement and variation of their conductive properties in the presence of targets. The plasmonic properties of gold and silver nanostructures include a unique tunability of the plasmon resonance properties of metal nanoparticles through variation of their size, shape, composition, and medium to allow nanostructures designed for specific bio-applications. Known nanostructure geometries, including nanorods, nanoshells, and nanoparticle pairs can exhibit dramatically enhanced and tunable plasmon resonances, making them highly suitable for bio-applications including detection and diagnostics. Tuning the nanostructure shape (e.g., nanoprisms, nanorods, or nanoshells) is another means of enhancing the sensitivity of the localized surface plasmon resonance (LSPR) to the biological sample and target entity. Metal nanoparticle pairs or assemblies display distance-dependent plasmon resonances as a result of field coupling. A universal scaling model, relating the plasmon resonance frequency to the interparticle distance in terms of the particle size, becomes potentially useful for diagnostics of conditions within biological systems.

The examples of the applications of noble metal nanostructures provided herein can be readily generalized to other areas of biology and medicine because plasmonic nanomaterials exhibit great range, versatility, and systematic tunability of their optical attributes. The fields that have recently been greatly impacted by the advancement in nanostructured materials are biology, biophysics, and medicine. The nanobiology toolkit has been greatly enhanced by noble metal nanostructures, which have proven to be highly versatile and tunable materials for a range of bioapplications including biophysical studies, optical properties of noble metal nanostructures and discuss recent research advances in their bioapplications.

Metal nanoparticles can be conjugated with small molecule or biomolecular targeting or recognition ligands for achieving molecular specificity. Each metal nanoparticle can be considered an optical probe equivalent to up to a million dye molecules. This provides a large margin for enhancing the probing sensitivity. Unlike dyes, metal nanoparticles are photostable and do not undergo photobleaching, allowing higher light excitation energies and longer probing times. There is a range of enhanced radiative and nonradiative attributes associated with the LSPR. The optical probing strategy can thus be chosen depending on the specific biological application. Different strategies may also be combined. Another unique property of LSPR is that it can be tuned by changing the nanostructure size, shape, composition, or environment in order to suit the bio-application. Gold nanoparticles in the 10-nm size range have a strong absorption maximum around 520 nm in water. This occurs around 390 nm for silver nanoparticles.

The kit can include a device component for measuring an amount of plasmonic resonance in a mixture of the biological sample compared to a control amount of aggregated noble metal nanoparticle for quantification of the target entity. In the event that the entity is detected, the device can also include quantification of an amount of entity in the sample. Quantification will typically be indicated by an increased intensity of color in the presence of a higher amount of the entity as compared to a control amount. Quantitation may also be achieved by detecting how many nanoparticles are bound to the target. Quantification is useful where the device is for monitoring the progression or regression of a condition. Typically, for example, an individual may want to learn whether a particular treatment is working. Whether the treatment is working may be known by detecting a reduced titer of virus particles per volume of body fluid measured. Identifying from a window in the device an indicator of a plasmonic resonance frequency of aggregated noble metal particles as compared to a plasmonic resonance frequency of non-aggregated noble metal particles in a control solution, wherein aggregation of the particles mixed with the sample indicates that the sample is positive for the entity. The assay further comprises measuring an amount of plasmonic resonance from aggregation compared to a control amount of plasmonic resonance from non-aggregated particles to quantitate an amount of entity in the sample.

An assay conducted within a hand-held device can collect a sample to be tested from an individual and within that sample can detect the presence of an entity. The assay can include pre-loading a chamber of the hand-held device with a detection solution of noble metal nanoparticles with ligands attached to their outer surfaces. The character of the ligands includes that they are specific for a moiety on the entity to be detected by the assay. The sample is collected from an individual and placed in a reservoir of the device. The sample is mixed with the pre-loaded detection solution and the entity is allowed to contact the analyte nanoparticles having ligands specific for the moieties on or within target entities. The method of conducting the assay can comprise drawing a blood sample into a test device, or allowing blood to drip from a finger prick into a collection chamber. From the collection chamber the blood can be filtered and the plasma isolated by passing the fluid through a membrane built into the device. Pressure and vacuum can be used to move the plasma to an assay chamber from the collection chamber. In the assay chamber the sample can contact reagents to perform the assay.

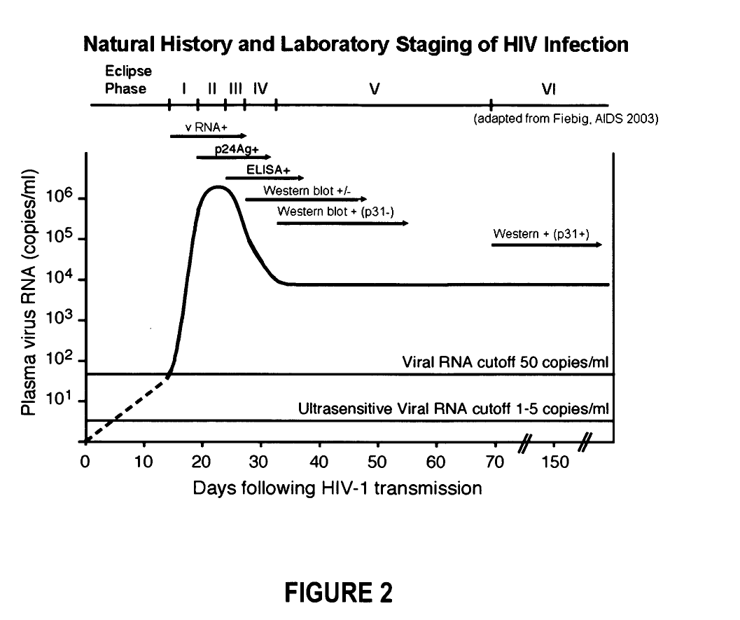

To exemplify the invention as a whole, a testing device, kit, assay and method are described for detecting HIV infection in humans. The life-threatening infectious disease known as acquired immune deficiency syndrome or AIDS is caused by infection with human immune-deficiency virus or HIV. Early detection of infection with HIV may provide greater success in HIV treatment by reducing the viral load during or just after the eclipse phase within the first 10 days of HIV infection (see FIG. 2). The invention provides the tremendous advantage of an ability to detect the human immune deficiency virus within the first 5 to 7 days of infection. Current testing methods for HIV cannot detect HIV until about 6 months after infection. In addition, infants suspected of contacting HIV, cannot be tested for antibodies until after one year of age. Thus, the deficiency of the current tests for HIV is profound. The current tests use the antibodies to HIV as the target entity for detecting HIV infection. The present invention is adapted to use a number of different target entities that will indicate the presence of HIV in a biological sample as soon as about 5 days after infection. For the purpose of the assay to test for HIV, the biological sample can be, for example, blood, semen, vaginal fluid, breast milk, other body fluids containing blood. Only specific fluids (blood, semen, vaginal secretions, and breast milk) from an HIV-infected person can transmit HIV. HIV is found in varying concentrations or amounts in blood, semen, vaginal fluid, breast milk, saliva, and tears.

The components of the HIV viron that can serve as target entities in the assay include, for example, the coat protein gp120, the RNA genome or sequences of the HIV RNA, or the capsid protein p24. For example, the invention can use ligands to detect RNA from HIV, p24 protein that forms the capsid encasement of the HIV genome, or ligands that recognize and bind regions of the coating protein of HIV gp120. Other target entities of HIV such as other proteins, enzymes or materials can also be used as target entities for the assay to detect HIV. Ligands to different components of the target entity can be used, increasing the likelihood of detection and strength of a positive signal. Different ligands for the same target entity binding different components of the target entity may be designed and used together in the assay to optimize the detection of a target. In the case of HIV, for example, the HIV viron or components of the HIV viron can be the target entity. Thus, the RNA genome of HIV, a component of the HIV viron, can be the target entity, and ligands specific for binding a region of the RNA genome can be used to detect the virus. Alternatively, different ligands specific for binding different sequences or regions of the RNA genome can be used. Also, for example, ligands specific for other components of the HIV viron can be used in addition, such as ligands specific for the capsid protein p24, and ligands specific for the gp120 coat protein.

With regard to the consideration of timing and the selection of a target entity, and specifically with regard to detecting HIV the assay can be conducted using a target entity that is present in the blood of an infected individual within about 5 days after infection. Prior and currently available tests for HIV detect the antibodies to HIV, and these antibodies do not appear in an infected individual until about 6 months after infection. Antibodies to HIV do not appear in an infected infant until 12 months after infection. Accordingly, target entities that can be used in the assay to detect HIV include the HIV viron or components of the viron that are present at very low levels in blood of an infected individual within about 5 days after infection.

To address the challenge of a test device, test kit, assay and method for detecting HIV in a biological sample from an individual collected into the test device, aspects of the structure, activity, and biology of HIV should be understood and considered. Instructive to understanding the structural and behavioral nature of HIV infection in humans, and in understanding aspects that may affect diagnosis and treatment reference is made to the article “Dynamics of HIV viremia and antibody seroconversion in plasma donors: implications for diagnosis and staging of primary HIV infection”, Fiebig et al., AIDS 2003, 17:1871-1879 for staging HIV, in which load concentrations during primary HIV infection is analyzed and discussed. Feibig et al. is incorporated by reference in its entirety.

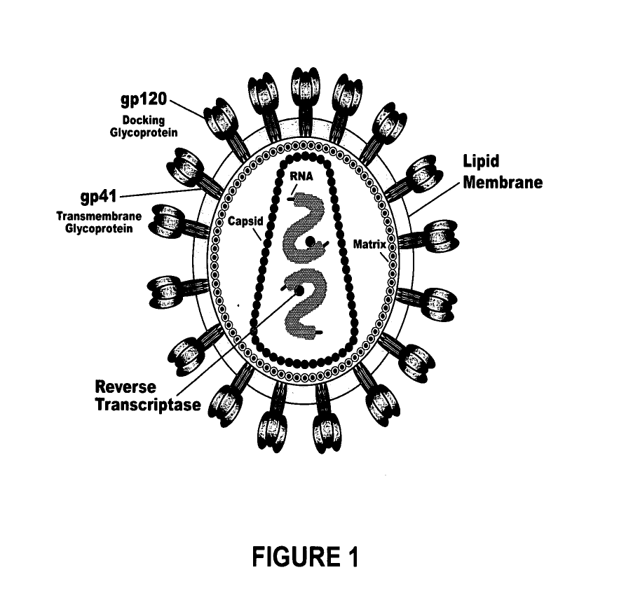

The biology of HIV in the body is pertinent here. Released HIV particles display a broad range of diameters, extending from 120 to 200 nm. The majority of virions on HIV have a single core. HIV virons are coated with 72 spikes of a trimeric protein assembly of gp120 anchored with gp41 proteins to the viral surface, FIG. 1. The viral core (or capsid) is made from the protein p24. Inside the core are three enzymes required for HIV replication called reverse transcriptase, integrase and protease. Also held within the core is HIV's genetic material, which consists of two identical strands of RNA. HIV is a retrovirus and its genes are composed of RNA (Ribonucleic Acid). HIV has nine genes: gag, pol, env, tat, rev, nef, vif, vpr and vpu. A viron of about 150 nm diameter will have a spherical surface area of about 70,685 nm² on which reside the 72 spikes. There are many possible markers of HIV, including antibodies to gp120 and p24, and the HIV RNA sequence. The RNA-based HIV detection test of this invention has an approximate sensitivity of about 1 copy of HIV per drop of blood in order to detect HIV infection within about 5 days after suspected contact. Currently, no plasma-based very low viron assays are FDA approved for the diagnosis of HIV-1 infection, and any early tests that might be possible at 15 days or later after infection, are not possible outside a medical facility.

In the case of a solution to detect HIV, one target entity on the HIV viron can be gp120 because the viron is coated with gp120 proteins. Antibodies specific for a portion of the gp120 protein on the viron surface can be conjugated to gold nanoparticles. An HIV viron having Surface Area=4 pi r² where r=½ the diameter of about 150 nm or r=about 75 nm has a surface area about=4×3.14×75²=70685 nm². The surface area of a nanoparticle of say for example, 15 nm diameter=4×3.14×7.5²=706.5² nm. Dividing the surface area of the viron by the surface area of a nanoparticle of 15 nm diameter seems to provide binding space for 100 nanoparticles on the viron surface. As there are 72 gp120 spikes, each spike having 3 gp120 proteins, taking a conservative approach to anticipate steric issues, etc. somewhere between 20 and 60 nanoparticles could bind a single HIV viron creating a massive nanoparticle cluster that would clearly indicate a shift from red color to blue color in the visible light spectrum. If the distance between nanoparticles is less than the diameter of any one nanoparticle, the plasmon shift represented in the visible spectrum occurs. Thus, the smaller the nanoparticles, the closer they must be to each other when binding their targets in order to facilitate the color shift. The principles of plasmonic resonance and color shifting rely on the aggregation of the nanoparticles, a low level of target analyte can be compensated for by making sure sufficient nanoparticles of an appropriate diameter conjugate to a plentiful target on the viron such that the nanoparticles are closer to each other than the diameter of the nanoparticles. An additional factor influencing the sensitivity of the assay is also how many ligands are conjugated to each nanoparticle. Nanoparticles are coated with sufficient but not excess binding ligands in order to facilitate binding to components of the HIV viron. Finally, detection using noble metal nanoparticle conjugates can be optimized with an assay technique called “crowding”. Crowding is the addition of bulk molecules to a solution to create space between entities in the solution and to also move entities in the solution closer together. Accordingly, optimization of detection can be achieved in the assay with the addition of known macromolecules such a polyethylene glycol (PEG) of 8 kilodaltons (8 K), PEG 20 K, PEG 35 K, Ficoll 70, Ficoll 400, Dextran 70 K, Dextran 500 K, Dextran 2000 K, and others like agents. See BMC Biotechnology 2011, 11:50 doi:10.1186/1472-6750-11-50. Also see Hill C S: Molecular diagnostic testing for infectious diseases using TMA technology. Expert Rev Mol Diagn 2001, 1(4):445-455. Both references are incorporated into this application in their entirety.

Detecting HIV can be accomplished by first contacting the biological sample with a lysing agent to lyse the viron and the viral capsid. The lysing agent can be, for example, guanidine thiocyanate. A lysing agent will release the mRNA from the capsid, and also the protein that forms the capsid p24. Nanoparticles conjugated to ligands comprising complementary sequences of the HIV genome can then bind HIV RNA (through the conjugated ligand) along the length of the RNA and potentially a single viron having two RNA strands will be detectable using colorimetric nanoparticle-based detection. An aggregrate of at least 60 nm diameter, and preferably multiple such aggregates form at the HIV RNA and shift the red color of the solution to a blue color. Optimally, in order that the entire RNA length is used for targeting by nanoparticle in solution, the nanoparticles can be conjugated to different sequences complementary to sequences on the RNA genome to increase the number of nanoparticles that come together in the solution. For details on potential target sequences within the HIV viron that may be used to form complementary ligands to bind the viron in an assay based on colorimetric nanoparticle detection see Moore M D, Nikolaitchik O A, Chen J, HammarskjöId M-L, Rekosh D, et al. (2009) Probing the HIV-1 Genomic RNA Trafficking Pathway and Dimerization by Genetic Recombination and Single Virion Analyses. PLoS Pathog 5(10): e1000627. doi:10.1371/journal.ppat.1000627. The Moore et at reference is incorporated in its entirety herein. Note also that plasma specimens taken from an individual can be lysed, and RNA can be stabilized and captured on magnetic particles containing poly(dT) oligonucleotides and oligonucleotides complementary to the viral RNA.

A test device and kit for detecting an infectious agent can have a means or component to ensure that the live virus is inactivated. In the case of HIV, known to have very feeble to no ability to infect individuals if not transmitted within live cells, and infection with a viron found outside the host body is unlikely. However, the design of a test device can include a feature to neutralize live virus after completion of the assay. As HIV is very sensitive to changes in alkalinity or acidity and thus pH levels below 7 or above 8 are unsuitable for long-term survival of HIV, a simple inactivation strategy for the virus can be accomplished before, after or during the assay by addition of acid or base. Solvent/detergent (S/D) inactivation, developed by the New York Blood Center, is the most widely used viral inactivation method to date. It is predominantly used in the blood plasma industry, but the process is only effective for viruses enveloped in a lipid coat. The detergents used in this method interrupt the interactions between the molecules in the virus's lipid coating. Most enveloped viruses cannot live without their lipid coating, so they die when exposed to these detergents. Other viruses may still live, but they are unable to reproduce, rendering them non-infective. The solvent creates an environment in which the aggregation reaction between the lipid coat and the detergent happen more rapidly. The detergent typically used is Triton-X 100.

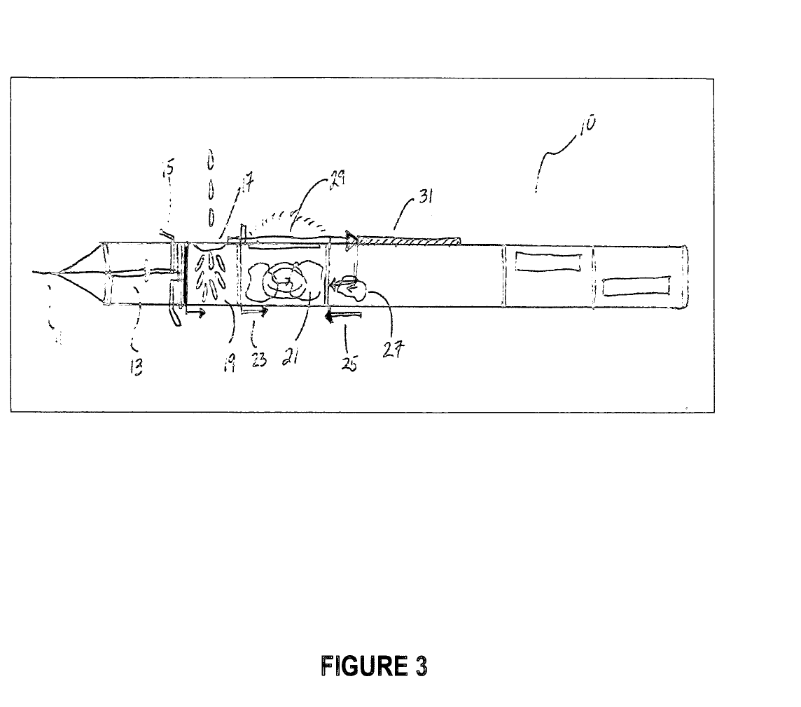

The device can also be designed so that in order to read the results of the assay, the chamber that holds the biological sample must be closed and sealed shut. This can be accomplished by having a cover positioned over the results window that is slid to close and seal the collection chamber and at the same time revealing the results window. Turning to FIG. 3, a schematic representation is shown of a testing device 10 having a tip and possible lancet 11 for opening the skin on a fingertip. Control bar 13 can push out the needle or lancet 11 using handle 15. Collection chamber 19 having opening 17 provides for collection of the sample. Filter and draw mechanism 23 pulls the sample through the filter 23 to contact and be mixed with analyte 21 in assay chamber 29. Optionally, cover 29 can be slid to position 31 after inactivation solution 27 is placed in the assay chamber to inactivate any live virus. As cover 29 is slid to the right (in the Figure), inactivation solution is moved to the assay chamber, and results window under cover 29 is opened to view the results of the assay.

Since the HIV concentrations used in laboratory studies are much higher than those actually found in blood or other specimens, drying of HIV-infected human blood or other body fluids reduces the theoretical risk of environmental transmission to that which has been observed—essentially zero. Additionally, HIV is unable to reproduce outside its living host (unlike many bacteria or fungi, which may do so under suitable conditions); therefore, HIV does not spread or maintain infectiousness outside its host. However, as a precautionary measure, for the HIV test kit and testing device, there can be a chemical or physical means to inactivate any still active live virus in the biological sample.

Other aspects of details of conducting the assay, including details about the best reagents to use, the best concentrations of these reagents in solution, how to conjugate ligands to the nanoparticles, selection of appropriate ligands, quantification principles, size considerations for the nanoparticles, optimal volume of solute in which the assay is conducted, considerations in generating a color that is sufficiently strong and visible in the results window, and other such details may be found in one or more of the following journal articles. Fox, Matthew. “Accuracy of the Elisa HIV May 2010. Web. <http://www.livestrong.com/article/133176-accuracy-elisa-hiv-test/>., Shah, I., et al. 2006. Efficacy of HIV PCR Techniques to Diagnose HIV in Infants Born to HIV Infected Mothers—An Indian Perspective. JAPI 54: 197-199, “What Kinds of HIV Screening Tests Are Available in the United States?” HIV InSite. UCSF, 3 Aug. 2011. Web. http://hivinsite.ucsf.edu/insite?page=basics-01-01, Xu, J., et al. 2010. Highly soluble PEGylated pyrene-gold nanoparticles dyads for sensitive turn-on fluorescent detection of biothiols. Analyst 135: 2323-2327, Lee, H., et al. 2009. Modeling sequence evolution in acute HIV-1 infection. Elsevier 261: 341-360, Kim, Y., et al. 2009. Quantum dot-based HIV capture and imaging in a microfluidic channel. Biosens Bioelectron 25: 253-258, Wang, S., et al. 2010. Advances in developing HIV-1 viral load assays for resource-limited settings. Biotechnology Advances 28: 770-781, Ahn, Chong H. January 2004. Disposable smart lab on a chip for point-of-care diagnostics. IEEE, Vol 92, No. 1, Fend, Yanying. June 2003. Passive valves based on hydrophobic microfluidics. Mems Laboratory, Rosina, J. Temperature Dependence of Blood Surface Tension. Physiological Research Pre-Press Article, Bush, Valeria and Richmond Cohen. 2009. The Evolution of Evacuated Blood Collection Tubes. LabNotes. Volume 19, No. 1, Winter, Jessica. “Gold Nanoparticle Biosensor.” Ohio State University. 23 May 2007. Web http://www.nsec.ohiostate.edu/teacher_workshop/Gold_Nanoparticles.pdf, Stowell, Dan. “The Molecules of HIV: gp120.” www.mcld.co.uk. 2006. Web. <http://www.mcld.co.uk/hiv/?q=gp120>. The articles cited above are all incorporated by reference in their entirety herein for use in supporting details for performing the assay on a biological sample contained in a test device after collection of the sample from an individual.

The scope of the invention is provided by the broadest interpretation of the language and details described here. The details presented here are intended to exemplify the invention, and not to limit it.

We claim:

1. A home testing device for testing the presence of a pathogen in a patient wherein removal of biological material and an assay to test said biological material for said pathogen is conducted in a single device, said device comprising a unit for receiving biological material and a chamber for testing for the presence of a target entity comprising a biological molecule in said material, wherein the presence of said biological molecule confirms the presence of said pathogen, wherein said chamber comprises an assay molecule capable of detecting the presence of said target entity comprising a biological molecule.

2. The device of claim 1, wherein the pathogen is HIV.

3. The device of claim 1, wherein the target entity comprising a biological molecule is a component of an HIV viron.

4. The device of claim 1, wherein the assay molecule is a nanoparticle.

5. The device of claim 4, wherein the nanoparticle is a noble metal nanoparticle.

6. The device of claim 5, wherein the noble metal nanoparticle is gold.

7. The device of claim 1, wherein the chamber comprises sufficient assay molecules to detect sufficient target entities comprising biological molecules in an amount of HIV virons present in a recently infected patient.

8. The device of claim 7, wherein the patient is infected about 30 days or less before conducting a home test using said home testing device.

9. The device of claim 7, wherein the device is capable of detecting multiple target entities comprising multiple biological molecules present in a few HIV virons in said biological material taken from the patient.

10. The device of claim 9, further comprising a safe closure to prevent contamination of biological material with another individual after removing said biological material from the patient.
